(19) United States
(12) Patent Application Publication
(10) Pub. No.: US 2012/0204881 A1
(43) Pub. Date: Aug. 16, 2012

(54) PAD FOR A MASK

(75) Inventors: Aaron Samuel Davidson, Mona Vale (AU); Gerard Michael Rummery, Woodford (AU); Justin John Formica, Voyager Point (AU); Adam Barlow, Manly (AU)

(73) Assignee: ResMed Limited, Bella Vista (AU)

(21) Appl. No.: 13/369,640

(22) Filed: Feb. 9, 2012

Related U.S. Application Data
(60) Provisional application No. 61/441,058, filed on Feb. 9, 2011.
(60) Provisional application No. 61/552,055, filed on Oct. 27, 2011.

(51) Int. Cl. A61M 16/06

(52) U.S. Cl. 128/206.25

(57) ABSTRACT

A cushion pad for a mask worn by a patient, includes a first side to contact skin of the patient, and a second side, opposite the first side, to contact or face the mask. The pad has a main body portion an upper attachment portion extending from a top of the main body portion, and a lower attachment portion extending from a bottom of the main body portion, the upper attachment portions adapted to attach to an upper portion of the mask and a the lower attachment portion adapted to connect to a lower portion of the mask.

### CROSS REFERENCE TO RELATED APPLICATION

This application claims the benefit of U.S. Provisional Application Nos. 61/441,058, filed Feb. 9, 2011, and 61/552,055 filed Oct. 27, 2011, incorporated herein by reference in its entirety.

### FIELD OF THE INVENTION

The present invention relates to a pad for a respiratory mask for the delivery of respiratory therapy to a patient. Various respiratory conditions include Sleep Disordered Breathing (SDB) and particularly Obstructive Sleep Apnea (OSA). Respiratory therapies used to treat these conditions include Continuous Positive Airway Pressure (CPAP), Non-Invasive Positive Pressure Ventilation (NIPPV), and Variable Positive Airway Pressure (VPAP).

### BACKGROUND OF THE INVENTION

Typically, respiratory therapy is delivered in the form of a respiratory mask or mask system positioned between a patient and apparatus providing a supply of pressurized air or breathing gas. Mask systems in the field of the invention differ from mask systems used in other applications such as aviation and safety in particular because of their emphasis on comfort. This high level of comfort is desired because patients must sleep wearing the masks for hours, possibly every night for the rest of their lives. In addition, therapy compliance can be improved if the patient's bed partner is not adversely affected by the patient's therapy and wearing of the mask generally.

Mask systems typically, although not always, comprise (i) a rigid or semi-rigid portion often referred to as a shell or frame, (ii) a soft, patient contacting portion often referred to as a cushion, and (iii) some form of headgear to hold the frame and cushion in position. If the mask system does include multiple components, at least some assembly and adjustment may be required, which can be difficult for patients who may suffer from lack of dexterity, etc. Further, mask systems often include a mechanism for connecting an air delivery conduit. The air delivery conduit may preferably be connected to a blower or flow generator.

A range of mask systems are known including nasal masks, nose & mouth masks, full face masks and nasal prongs, pillows, nozzles & cannulae. Masks typically cover more of the face than nasal prongs, pillows, nozzles and cannulae.

Some respiratory masks cause discomfort or are difficult to seal on some patients. Comfort and seal may be particularly troublesome in the nasal bridge region. Other portions of the mask system including but not limited to the forehead support and headgear clips may cause discomfort or cause skin breakdown (e.g. ulceration, skin marking, irritation, redness, etc).

### SUMMARY OF THE INVENTION

One aspect of the present technology relates to a pad for a mask system.

Another aspect of the present technology relates to a pad for a mask system with an adhesive. In another aspect, no adhesive is used. In another aspect, the pad may include a tacky or adhesive portion.

Another aspect of the present technology relates to a pad for a mask system, the pad comprising a tacky gel material.

Another aspect of the present technology relates to a pad for a mask system, the pad being adapted to connect and disconnect from the mask system.

Another aspect of the present technology relates to a pad for a mask system, the pad being adapted to connect with a forehead support of the mask system.

Another aspect of the present technology relates to a pad for a mask system, the pad being adapted to connect with an elbow of the mask system.

Another aspect of the present technology relates to a pad for a mask system, the pad being adapted to connect with an elbow and a forehead support of the mask system.

Another aspect of the present technology relates to a cushion pad for a mask worn by a patient. The cushion pad includes a first side to contact the skin of the patient, and a second side, opposite the first side, to contact or face the mask, the pad having a main body portion, an upper attachment portion extending from the top of the main body portion, and a lower attachment portion extending from the bottom of the main body portion, the upper attachment portions adapted to attach to an upper portion of the mask and the lower attachment portion adapted to connect to a lower portion of the mask.

Another aspect of the present technology relates to a cushion pad for a mask worn by a patient. The cushion pad includes a pad having a central region and an edge region defining an outer perimeter that substantially surrounds the central region, wherein the central region has a first level of adhesiveness, and the edge region has a second level of adhesiveness different from the first level of adhesiveness.

Another aspect of the present technology relates to a cushion pad for a mask worn by a patient. The cushion pad includes a main body portion to cover a nasal bridge or another region of a patient, and a material provided to at least a portion of the main body, the material being provided in a pattern or layer on the main body to adhere the main body to the wearer or the mask in use, said pattern or layer providing a plurality of open regions on the main body that are not provided with said material.

Another aspect of the present technology relates to a cushion pad for a mask worn by a patient. The cushion pad includes a first side to contact skin of the patient, and a second side, opposite the first side, to contact or face the mask, the pad having a main body portion, and a flap-like portion extending downward from the main body portion, the flap-like portion adapted to protect the bridge of a nose of a user from contacting the cushion of the mask.

Another aspect of the present technology relates to a mask comprising a cushion and the cushion pads described above.

Another aspect of the present technology relates to methods of manufacturing a mask pad, e.g., via thermoforming, molding, machining, stitching, die cutting and/or compression cutting.

Other aspects, features, and advantages of the present technology will become apparent from the following detailed description when taken in conjunction with the accompanying drawings, which are a part of this disclosure and which illustrate, by way of example, principles of this technology.

### BRIEF DESCRIPTION OF THE DRAWINGS

The accompanying drawings facilitate an understanding of the various embodiments of the technology. In such drawings:

FIG. 1 is a perspective view illustrating a mask with a pad on a patient according to the present technology;

FIG. 2 is a side of the mask with a pad of FIG. 1;

FIG. 3 is a front view of the mask with a pad of FIG. 1;

FIG. 4 is a rear view of the mask with a pad of FIG. 1;

FIG. 5 is a front view of the pad of FIG. 1;

FIG. 6 is a front view of a pad for use with a mask according to another embodiment of the present technology; and

FIG. 7 is a front view of a pad for use with a mask according to another embodiment of the present technology.

### DETAILED DESCRIPTION

The following description is provided in relation to several examples which may share common characteristics and features. It is to be understood that one or more features of any one example may be combinable with one or more features of the other examples. In addition, any single feature or combination of features in any of the example or examples may constitute an additional feature which applicants may opt to independently protect.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

The term “air” will be taken to include breathable gases, for example air with supplemental oxygen. It is also acknowledged that the PAP devices or blowers described herein may be designed to pump fluids other than air.

### 1.0 Mask System

A range of masks (also referred to as patient interfaces or mask systems) are known including nasal masks, nose & mouth masks, full-face masks and nasal prongs, pillows, cradles, nozzles & cannulae.

A mask system typically includes a frame, a cushion and a headgear. The frame may anchor the cushion in position and allow for attachment of headgear. The frame is typically a rigid or semi rigid component. The cushion may seal with the face of the patient in order to provide therapy to the patient. The cushion is typically a flexible element. The headgear may stabilize and support the frame and cushion in position on the patient's face when in use. The headgear is typically a flexible or semi rigid element constructed of, for example, textile (e.g. fabric).

Some patients may have difficulty attaining a seal when the mask system is in position on the patient's face (both dynamic and static) for several reasons, such as unique facial profiles or facial hair. Some patients may experience discomfort, such as irritation due to prolonged contact with the mask system, particularly the cushion. In addition, if the patient has damaged skin that may contact the mask system, the patient may need a pad between the mask system and the patient's face to reduce pressure and enhance healing at the damaged skin site when the mask system is next used. A pad may also reduce rubbing of the patient's hair and thereby reduce hair loss. A pad may also assist in sealing over facial hair as it may more readily conform over the facial hair to create a seal between the patient and the mask system.

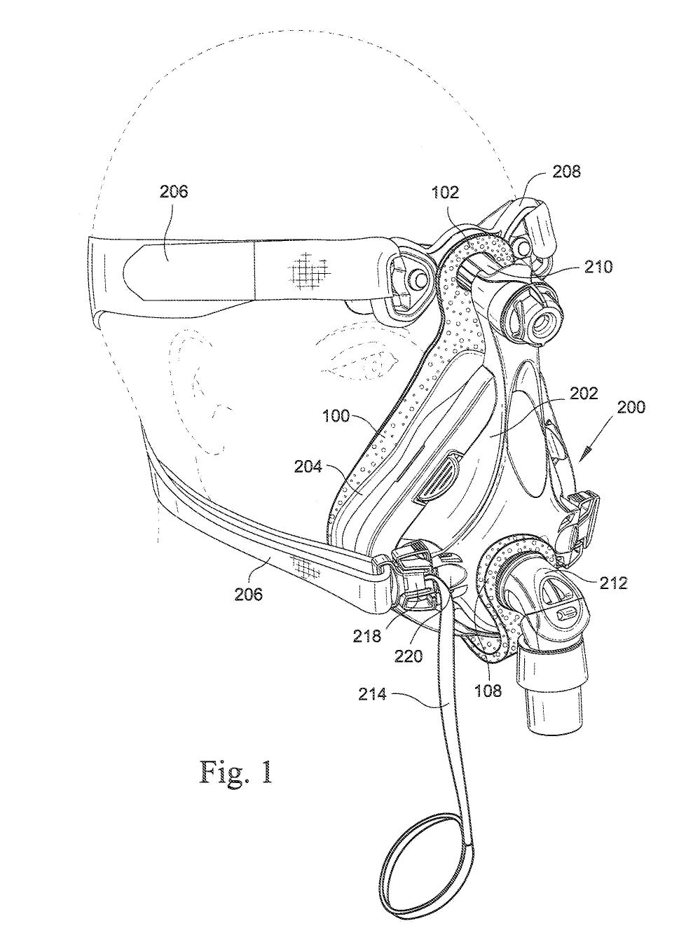

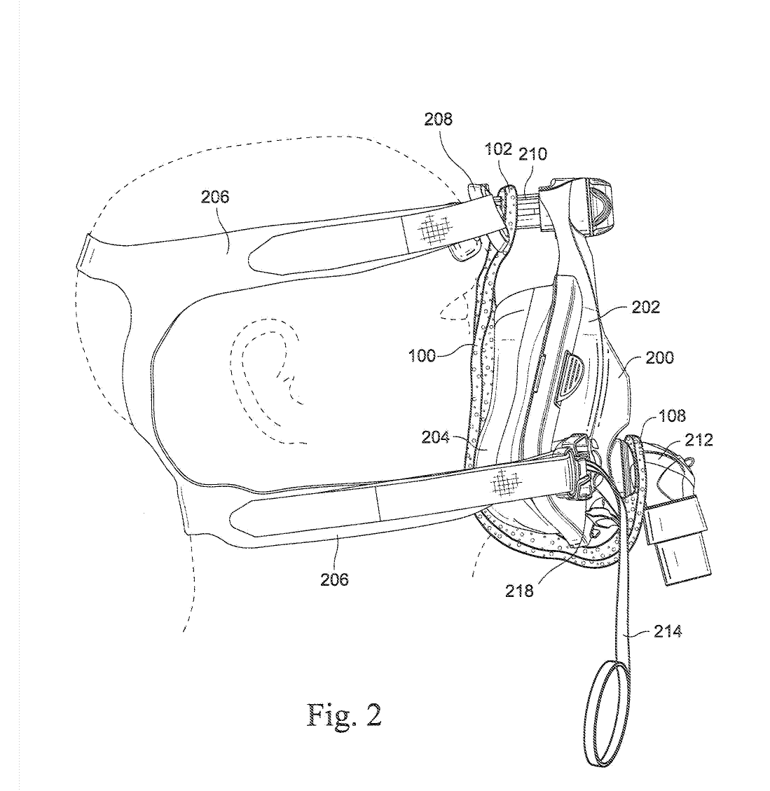

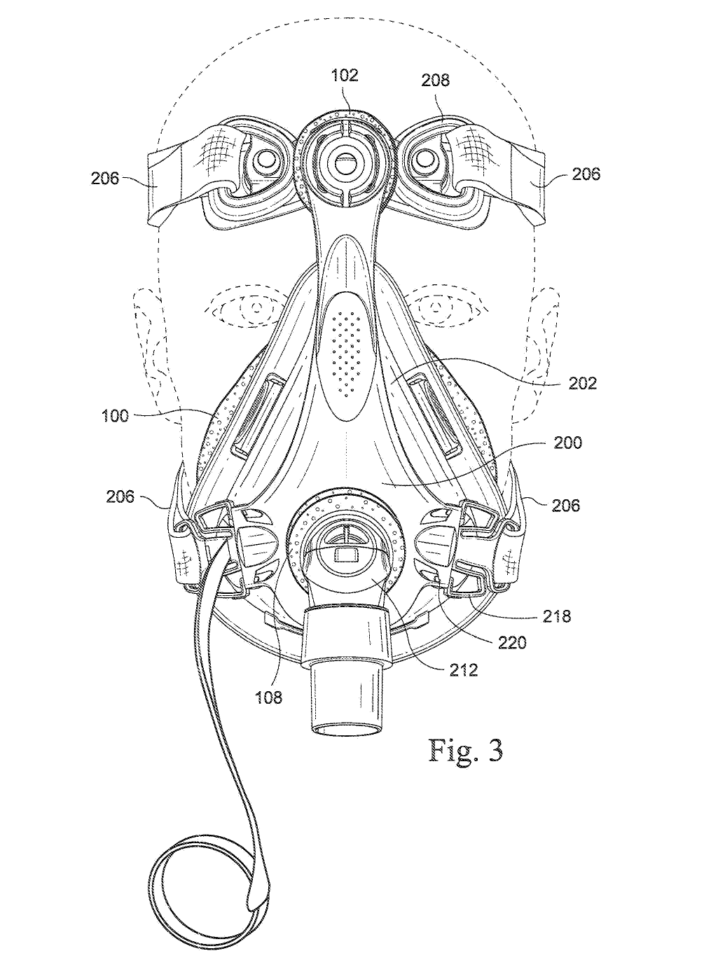

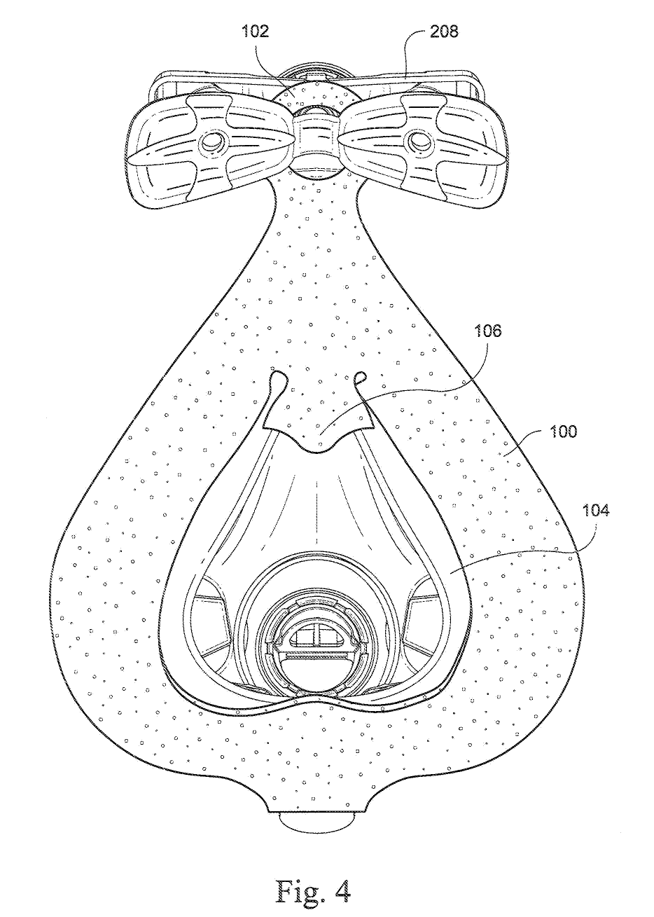

FIGS. 1-4 illustrate a mask system 200. The mask system 200 includes a frame 202, a cushion 204 and headgear 206. The frame 202 anchors the cushion 204 in position. The frame 202 may be a rigid or semi rigid component constructed of, for example, polycarbonate. The cushion 204 is typically a flexible element constructed of, for example, silicone to provide a comfortable fit to the patient.

The mask system 200 may include a forehead support 208, and an elbow 212. A post 210 may extend between the frame 202 and the forehead support 208. The elbow 212 includes a first end that interfaces with an aperture in the frame 202, and a second end adapted to connect to an air delivery tube that delivers pressurized breathable gas to the patient (in the range of 2-30 cm H₂O, typically 8-12 cm H₂O for OSA treatment). A pad 100 may be used with the mask system 200. The pad 100 may be placed between the cushion 204 and the patient's face.

The headgear 206 is adapted to secure the mask system 200 to the face of the patient. The headgear 206 may include headgear clips 218 adapted to connect to headgear connectors 220 on the frame 202. A quick disconnect element 214 may be provided that allows the headgear clips 218 to be quickly disconnected from the headgear connectors 220.

### 2.0 Pad

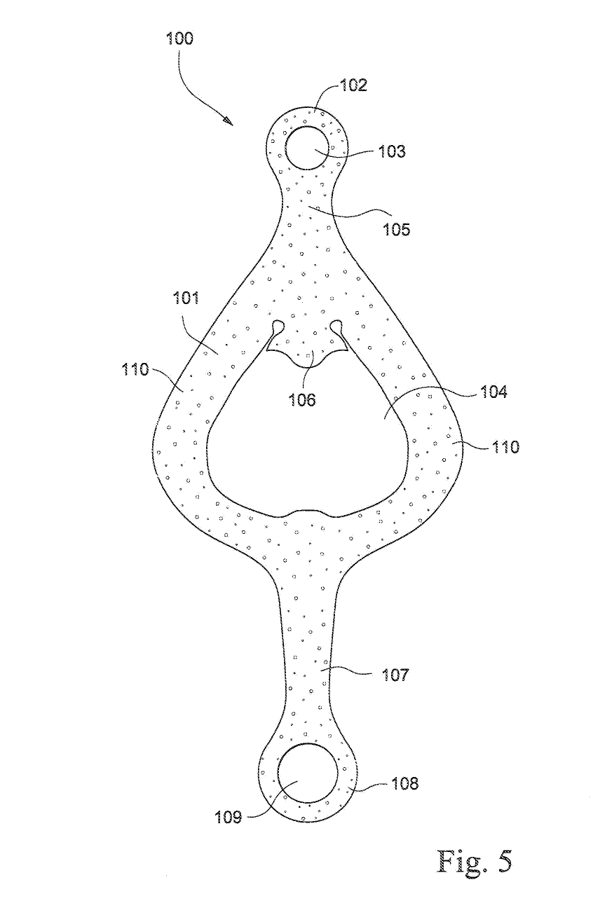

Pad 100 is illustrated in use with the mask system 200 in FIGS. 1-4, and is illustrated alone in FIG. 5. The pad 100 may include a main body portion 101 having side portions 110, an upper attachment portion 105 and a lower attachment portion 107. An alternative aspect of the technology may comprise a single attachment portion of the main body. The main body portion 101 has a shape that generally matches the shape of the cushion 204. Where the cushion 204 has a generally triangular shape, the main body portion 101 also has a generally triangular shape. The shape of the main body portion 101 may be altered from that shown to match the cushion shape of the mask that the pad is to be used with.

The main body portion 101 may include a nasal bridge portion 106 that extends downward from the apex of the generally triangular shape of the main body portion. The nasal bridge portion 106 may provide padding to the user's nasal bridge region. The nasal bridge portion 106 may have a lower flap-like portion. The lower flap-like portion may be provided to ensure that the patient's nasal bridge is protected from contacting the cushion 204. In particular, it is possible for the patient's nose to contact the cushion 204 when their nasal bridge is pressed into the cushion. For example, a patient with a high nose bridge may contact an undercushion of cushion 204. The flap-like portion may prevent this from occurring. The flap-like portion may include perforations, weakened regions or slits to permit it to flex or wrap about the main body portion 101. This is to allow the patient's nasal bridge to move into the cushion without disrupting the position of the main body portion 110. For example, a patient with a low nose bridge may not displace the flap like portion from the same plane as the main body portion 101. However, a patient with a comparably higher nose bridge may displace the flap like portion inwards, and with the slits, perforations or weakened regions, that flap like portion can freely displace inwards of the main body portion 101 without changing the position of the main body portion 101 or increasing the pressure on the patient's nose bridge.

The upper attachment portion 105 may extend upward from the apex of the main body portion 101 and may include an upper attachment portion 102, e.g., in the shape of a ring or a hook, or the like. An alternative aspect of the technology may include more than one upper attachment portion. The ring portion 102 may have include a portion having a generally circular shape (or any other shape that is suitable for attachment with the mask frame) with an aperture 103. The upper attachment portion 105 is adapted to connect the pad 100 to the mask system 200 at an upper portion of the mask system 200. For example, the upper attachment portion may connect to the forehead support 208, such as by connecting to the post 210 of the forehead support. This connection secures the upper portion of the pad 100 in relation to the mask system 200. The aperture 103 may be sized to fit around the post 210.

The aperture 103 may have a diameter of about 2 mm to 25 mm, or about 14 mm to 18 mm. The aperture 103 may have a diameter of about 10 mm to 24 mm. The ring 102 may have an outer diameter of about 40 mm to 44 mm. The ring 102 may have a circular opening, or another shape. It may also have a slit to facilitate assembly and disassembly to/from the post 210, or may connect to another portion of the forehead support 208 or to an upper portion of the frame. Instead of the ring 102, the upper attachment portion 105 may end with the ring, a hook portion or other shape that is adapted to connect to the forehead support 208 or to an upper portion of the frame 202. Upper attachment portion 105 may be formed of a stretchy or conformable material adapted to conform to the object it is attaching to.

The lower attachment portion 107 may extend downward from a base of the main body portion 101, and may include a ring portion 108. The ring portion 108 may have a generally circular shape (or any other shape suitable for attachment to a component of the mask) and includes an aperture 109. The lower attachment portion 107 is adapted to connect the pad 100 to the mask system 200 at a lower portion of the mask system 200. For example, the lower attachment portion 107 may extend below a bottom of the mask system 200, and be adapted to connect to a lower portion and/or a front side of the mask system 200.

The aperture 109 of the ring portion 108 may be sized to connect to the elbow 212. The aperture 109 may have a diameter of about 2 mm to 25 mm, for example about 12 mm to 24 mm. The aperture 109 may have a diameter of about 10 mm to 30 mm. The ring 108 may have an outer diameter of about 40 mm to 54 mm. The ring 108 may have a circular opening, or another shape. It may also have a slit to facilitate assembly and disassembly to/from the elbow 212, or may connect to another element on, or to a lower portion of the frame 202. Instead of the ring 108, the lower attachment portion 107 may end with a hook portion or other shape that is adapted to connect to the elbow 212 or to another element on, or to a lower portion of the frame 202. For example, the lower attachment portion 107 may connect to a port or ports on the lower portion of the frame 202, or may be adapted to connect to the headgear connectors 220.

The ring portion 108 may be connected to the elbow 212 by sliding the aperture 109 over the distal end of the elbow 212 while the elbow 212 is connected to the mask system 200, or by disconnecting the elbow 212 from the mask system 200, and inserting the end of the elbow 212 that connects to the mask system 200 into the aperture 109 before connecting the elbow 212 to the mask system 200. This connection secures the lower portion of the pad 100 in relation to the mask system 200.

The connection of the pad to the elbow 212 is particularly advantageous in systems where the elbow 212 is dis-connectable from the mask system 200, as such allows the user to quickly connect or disconnect the lower attachment portion 107 from the mask system. Further, utilizing the upper attachment portion 105 and the lower attachment portion 107 to secure the pad 100 to the mask system 200 enables the user to retain the pad 100 in position while connected to the mask system 200, and to easily disconnect the pad 100 if the user desires to use the mask system 200 without the pad 100.

The pad 100 is adapted to be placed between the cushion 204 and the face of the patient. Portions of the pad 100 may thus be positioned to be between the user's face and the cushion of the mask The pad may preferably be wider than the cushion of the mask so that no portion of the cushion of the mask will touch the user's face.

The pad 100 may also cover more the of nasal bridge region that tends to suffer the most discomfort or damage (since the top of the nose bridge is a small area, it is most susceptible to skin breakdown as the force from the mask system is distributed over a smaller area, also the skin can be thinner in this region when compared to other regions of the face e.g. cheeks) with the nasal bridge region.

Pad 100 may be constructed from a flexible material. The pad 100 or other pads herein may have one or more of the attributes of the pads described in pending U.S. patent application U.S. Ser. No. 12/801,307 filed Jun. 4, 2010, incorporated herein by reference in its entirety. Preferably pad 100 may be constructed from a foam such as that disclosed in pending US patent application US 2008/0047560, filed 27 Jul. 2007; pending US patent application US 2008/0060649, filed 27 Jul. 2007; pending U.S. patent application Ser. No. 12/448,250, filed 15 Jun. 2009, each incorporated herein by reference in its entirety. Other foams are also possible. Pad 100 may be constructed from textile (e.g. fabric) (e.g. woven or non-woven), gel, thermoplastic elastomer (TPE), foam or any other suitable material. Pad 100 may be constructed from a combination of materials, e.g., textile (e.g. fabric) and foam.

The pad may have a one piece or multiple piece construction. If multiple piece, it may include several layers, one of which is best suited to contact the patient, and the opposite which is suited to engage with the cushion. Moreover, one or more of the ring portions or fittings may be made of a different material than the main body of the pad, e.g., they can be made of an elastic material or different density foam, which is attached or otherwise mounted to the main body.

Pad 100 may be used with a full face mask (i.e. covering the nose and mouth of the patient), or with a nasal-only mask (i.e. cover or seal under the nose only), or with nasal pillows (prongs inserted into the patient's nose).

Advantages of the pad are that it may be disposable, intuitive to fit due to the shape and size, comfortable, enable a better or enhanced seal of the mask system when in use, and/or may encourage patient's to be compliant with therapy. The pad is ideally suited to allow both comfort and sealing in areas that are particularly sensitive, while avoiding application of extensive headgear strap tension to compensate for leaks, in which case unwanted pressure can be applied to other areas of the face, such as the cheeks and elsewhere. The pad may provide an improved seal especially with patients that have unusual facial shapes, beards/moustaches, and can assist with stability if the patient uses moisturizer or otherwise has greasy skin. The pad can also help avoid or dissipate uncomfortable leak around the patient's eyes.

Preferably, pad 100 may be formed by die cutting. Pad 100 may be formed by compression cutting. Pad 100 may be formed by molding (or co-molding). An adhesive may be provided to one or both sides of the pad. The adhesive may be laminated on to the foam before or after cutting the pad.

3.0 Pad with Adhesive Attachment

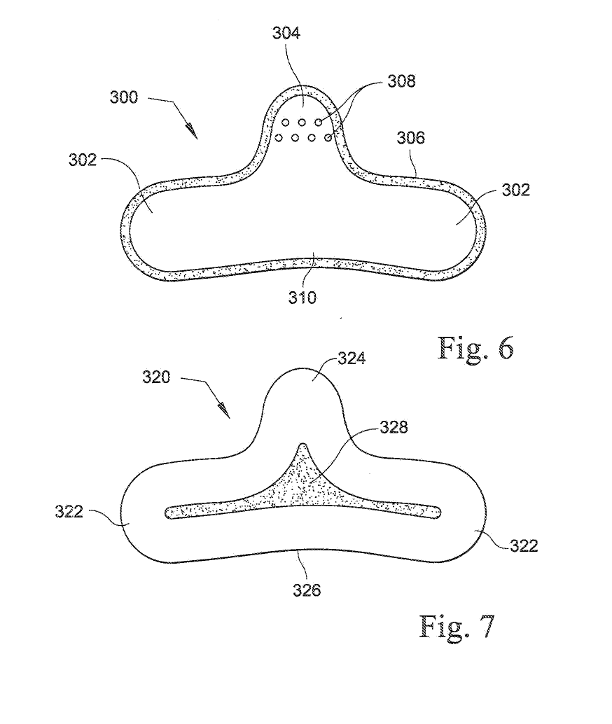

FIGS. 6 and 7 illustrate cushion or pads 300 and 320 that may be provided for use with a mask system such as mask system 200. Pads 300 and 320 are adapted to be placed on the face of a user prior to the user donning the mask, but also may be incorporated into the mask. The pads 300, 320 may be composed of a tacky gel material, such as a viscoelastic material, that is placed on the user's nose bridge region, as disclosed in US. Patent Publication 2010/0018535, filed Jan. 28, 2010, which is incorporated by reference herein in its entirety. The pad 100 may also be a foam or other material having little or no adhesiveness.

The pad 300 has a central region 304 having two side flaps or lobes 302. The central region 304 may be thickest, and taper down at the side lobes 302 to aid in sealing the pad with the mask, i.e., reduce the gap between the edge of the pad and the mask in use. Alternatively, the pad 300 may have a uniform thickness or a variable thickness.

The central region 304 of the pad 300 is adapted to be positioned on and extend upward from the bridge of the user's nose and absorb the force of the mask on the user's nose bridge. The side lobes 302 are adapted to extend laterally from the user's nose in use. The edges 310 of the pad may be tapered to avoid undue leakage where the pad ends.

The pad 300 may include one or more perforations or holes 308 that increase breathability of the pad. The holes 300 may be randomly disposed or aligned so that the pad may stick to different parts of the user's skin and not stick to other parts of the user's skin. The holes 308 may be selectively located in regions corresponding to sensitive regions of the patient's face, e.g., the nose bridge region.

An adhesive may be applied to the outer perimeter or edge region 306 of the pad 300 (or pad 100) to secure the pad 300 to the patient's face in use. The edge region may have a width of 1-40 mm, for example. The adhesive may be a skin compatible adhesive such as a silicone adhesive. The adhesive may be the same as those disclosed in pending U.S. patent application Ser. No. 12/478,537, filed 4 Jun. 2009, incorporated herein by reference in its entirety. The adhesive may be a low tack adhesive.

The adhesive may be covered with a release liner when not in use. The release liner may be kraft paper, silicone backed paper or any other suitable material.

As an alternative to the pad 300 being a tacky gel material, the pad 300 may be a foam pad, particularly if the adhesive is utilized. Also, adhesive may be applied to both sides of the pad, to adhere to the mask system and the patient.

Additionally, where the pad 300 is foam or a tacky gel material, the adhesive may be applied only to a selected portion of the pad, such as only at the outer perimeter or edge region 306 of the pad 300 with the central portion of the pad having less or no adhesive material.

FIG. 7 illustrates a pad 320 that has a central region 324 with two side flaps or lobes 322. The lower edge 326 of the pad 320 may be tapered to avoid undue leakage where the pad ends. The pad 320 may have any of the properties of pad 300. Additionally, the pad 320 may include an inner region 328. The adhesive or other tacky material, e.g, gel material, may be selectively applied only to the inner region 328, whether the pad 320 is a tacky material or a foam material. The pad 320 may be formed primarily from foam or another non-tacky material, with the inner portion 328 formed from a tacky material. By utilizing an adhesive or tacky material only on a portion of the pad such as the outer edge or inner region 328, occluding all of the user's skin underlying the pad can be avoided.

### 4.0 Pad Variations

The thickness of pad 100, 300, 320 may be constant along the length of the pad. The thickness of pad 100 may vary along its length. The pad 100, 300, 320 may have a thickness of 1 to 15 mm. The pad 100, 300, 320 may have an increased thickness at the nose bridge region, or may have an increased thickness at edges of the mask to aid in mask fit. The pad 100, 300, 320 may be available in multiple configurations to fit different mask systems. A portion of the pad 100, 300, 320 may have a contoured outer surface (e.g., concave or grooved or the like) to facilitate adjustment with and engagement of the cushion.

The pad 100, 300, 320 may include a surface texture applied to the bottom side (user contacting side) to permit some air to flow between the pad and the user's skin, thereby allowing the user's skin to breathe. The pad 100, 300, 320 may also include a surface texture applied to the top side (non-user contacting side) to increase the effectiveness of the seal with the mask and/or the visual appeal of the pad.

The pad 100, 300, 320 may include portions having varying hardness. For example, the pad 100, 300, 320 may include a portion or portions with a lower hardness than other portions, the lower hardness regions being used in areas of the pad that will be applied to more sensitive regions of the user's face. For example, the portion of the pad to be applied to the nasal bridge region of the patient may have a lower hardness than the portion of the pad to be applied to the patient's sides of the nose or cheek region.

Pad 100, 300, 320 may be constructed from a flexible, breathable material. Preferably pad 100, 300, 320 may be constructed from a foam such as that disclosed in pending US patent application US 2008/0047560, filed 27 Jul. 2007; pending US patent application US 2008/0060649, filed 27 Jul. 2007; pending U.S. patent application Ser. No. 12/448,250, filed 15 Jun. 2009. Other foams are also possible. Pad 100, 300, 320 may be constructed from textile (e.g. fabric) (e.g. woven or non-woven), gel, thermoplastic elastomer (TPE) or any other suitable material. Pad 100, 300, 320 may be constructed from a combination of materials e.g. textile (e.g. fabric) and foam.

The pad 100, 300, 320 may include a main body with a material provided to at least a portion of the main body, the material being provided in a pattern on the main body to adhere the main body to the user or the mask in use, said pattern providing a plurality of open regions on the main body that are not provided with said material. The pattern may be a random pattern, a checkerboard pattern. The pattern may include one or more grid portions spaced at substantially regular intervals. The pattern may be in the form of a grid or mesh, including a plurality of cross members. The pattern may include a plurality of portions that are spaced from one another, with the spaces between the portions not having said material. Alternatively, the plurality of portions may extend only in the longitudinal direction or along the longest axis of the pad. The plurality of portions may be elongated regions of the material, and they may extend generally parallel to each other. This may assist in removal of the pad as patients are more likely to peel the pad from their nose by gripping one lobe and pulling this lobe towards the other lobe of the pad. With the plurality of portions aligned along this direction, it may be easier for the patient to remove the pad. Alternatively, the plurality of portions may be aligned in a direction normal to that of the longest axis to provide greater stability of the pad on the superior-inferior direction of the patient's nose, i.e., to prevent the pad from slipping down the patient's nose.

The material provided in the pattern may include an adhesive, a gel, or the like. The material is provided to a central region of the main body, an edge region of the main body, or both the central region and the edge region of the main body. The main body has first and second opposed surfaces or sides (a user contacting side and a mask contacting side, one or both of which may include said material in the form of said pattern.

Pad 100, 300, 320 may be used with a full face mask (i.e. covering the nose and mouth of the patient), or with a nasal-only mask (i.e. cover the nose only).

Pad 100, 300, 320 may be thermoformed and/or die (or compression) cut. Pad 100, 300, 320 may be formed by thermoforming and ultrasonic die cutting. Alternatively, pad may be molded, for example by injection molding, compression molding, etc. In a further alternative, pad 100, 300, 320 may be machined and/or stitched. Pad 100, 300, 320 may be formed by any other suitable means.

The pad 100, 300, 320 may be used with a full face mask (i.e. covering the nose and mouth of the patient), or with a nasal-only mask (i.e. cover the nose only).

When all or a portion of the pad 100, 300, 320 is tacky, it may be tacky on one or both sides. This may enable the pad to engage first with the patient's face and then with the mask so as to ensure a stable seal between the patient and the mask. Alternatively, the pad may be first attached to the mask and then applied to the patient's face.

The pad 100, 300, 320 may be attached or molded directly to the mask. The pad 100, 300, 320 may be displaced wholly within the mask seal to reduce any problems of the mask sealing with the patient's face.

The pad 100, 300, 320 may be formed of a textile (e.g. fabric) material, with a gel or tacky material applied to the textile (e.g. fabric), so that there is a soft outer layer, i.e., mold a conformable material to the textile (e.g. fabric).

The pad 100, 300, 320 may be made from a spacer fabric and ultrasonically die cut so that the edges have a tapered finish. The spacer fabric could also be embossed for comfort and breathability.

The pad 100, 300, 320 may incorporate a nasal dilator to aid in therapy, such as disclosed in U.S. patent application Ser. No. 11/886,667 filed Mar. 10, 2006, which is incorporated herein by reference in it's entirety.

The pad 100, 300, 320 may be attached to the patient's face not by adhesive or other tacky material, but a malleable wire may be attached or molded to the pad so that the user can pinch the pad on to their nose.

The pad 100, 300, 320 may be a sack or bladder filled with a flowable material such as water that can more readily deform than an gel, for example.

Additives may be added to the pad 100, 300, 320 such as moisturizers or healing agents. These additives may diffuse through the pad and on to or into the patient's skin over a prolonged period of time. Alternatively, the additives may diffuse through the pad at a certain fixed time or over a short period of time.

The pad 100, 300, 320 may encourage the user's skin to cool down by including a phase changing material in the pad.

The pad 100, 300, 320 may include silver as an antibacterial. This may be particularly useful for treatment of pressure sores in diabetics, for example.

The pad 100, 300, 320 may include an anti-wicking agent to help prevent skin breakdown by removing moisture from between the skin and the pad.

The pad 100, 300, 320 may include a vapor transfer material that could be added to maintain a constant level of moisture in the skin.

The pad 100, 300, 320 may include a dual layer material (e.g., textile (e.g. fabric)) so that there is friction between the mask and the pad to increase seal between these elements, and comfort between the pad and the user.

The pad 100, 300, 320 may be electrostatic to encourage a seal with silicone masks. For example, the pad may be made from polystyrene, which is attracted to silicone by electrostatic forces.

Advantages of the pad are that it may be disposable, intuitive to fit due to the shape and size, comfortable, enable a better or enhance seal of the mask system when in use, and/or may encourage patient's to be compliant with therapy. Another advantage of a pad in accordance with the attachment and or tether aspects of the present technology is that it does not fall off the mask and get lost, or become soiled when the mask is removed from the face.

The pad 100, 300, 320 may be applied to the user's face after a flowable material is painted, smeared on or otherwise applied on to the sensitive regions of the patient's face. The material may react when applied to the face, e.g. foam. The material may disperse or run when applied to the face to cover more area and thereby protect more skin. The material may react with body heat or body fluid in order to cure. Alternatively the material may cure when heat or other substances are applied, e.g. a hair dryer or humidified air from the flow generator could cure the material after it is applied to the user's face. The material may be self skinning. The material may be a cream (e.g. barrier cream), latex (or equivalent), clay, gel, foam, polymer, etc.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention. Also, the various embodiments described above may be implemented in conjunction with other embodiments, e.g., aspects of one embodiment may be combined with aspects of another embodiment to realize yet other embodiments. Further, each independent feature or component of any given assembly may constitute an additional embodiment. Furthermore, each individual component of any given assembly, one or more portions of an individual component of any given assembly, and various combinations of components from one or more embodiments may include one or more ornamental design features. In addition, while the invention has particular application to patients who suffer from OSA, it is to be appreciated that patients who suffer from other illnesses (e.g., congestive heart failure, diabetes, morbid obesity, stroke, bariatric surgery, etc.) can derive benefit from the above teachings. Moreover, the above teachings have applicability with patients and non-patients alike in non-medical applications.

1-35. (canceled)

36. A cushion pad for a mask, comprising:
a pad having a central region and an edge region defining an outer perimeter that substantially surrounds the central region, wherein the central region has a first level of adhesiveness, and the edge region has a second level of adhesiveness different from the first level of adhesiveness.

37. The cushion pad according to claim 36, wherein the first level of adhesiveness is greater than the second level of adhesiveness.

38. The cushion pad according to claim 37, wherein the edge region has little or no adhesiveness.

39. The cushion pad according to claim 36, wherein the second level of adhesiveness is greater than the first level of adhesiveness.

40. The cushion pad according to claim 37, wherein the central region has little or no adhesiveness.

41. The cushion pad according to claim 36, wherein the thickness of the pad is 1-15 mm.

42. The cushion pad according to claim 36, wherein the width of the edge region is 1-40 mm.

43. The cushion pad according to claim 36, wherein the adhesive is a skin compatible adhesive.

44. The cushion pad according to claim 36, wherein the adhesive is a silicon adhesive.

45. The cushion pad according to claim 36, wherein the adhesive is a low tack adhesive.

46. The cushion pad according to claim 36, wherein the adhesive is covered with a release liner.

47. The cushion pad according to claim 36, wherein a location of the adhesive on the pad is randomized.

48. The cushion pad according to claim 36, wherein the pad comprises a foam material.

49. The cushion pad according to claim 36, wherein the pad comprises a gel material.

50. The cushion pad according to claim 36, further comprising a plurality of holes formed in the pad that increase breathability of the pad.

51. The cushion pad according to claim 50, wherein the holes are randomly disposed on the pad.

52. The cushion pad according to claim 50, wherein the holes are selectively located in regions corresponding to more sensitive regions of a patient's face.

53. The cushion pad according to claim 50, wherein the holes are located in a nose bridge region.

54. The cushion pad according to claim 36, wherein the pad has a surface texture to permit air flow.

55. The cushion pad according to claim 54, wherein the surface texture is on both sides of the pad.

56. The cushion pad according to claim 36, wherein the pad has a varying hardness.

57. The cushion pad according to claim 36, wherein the pad is attached or molded to the mask.

58. The cushion pad according to claim 36, wherein the pad comprises a textile (e.g. fabric) material with a tacky material applied to the textile (e.g. fabric) material.

59. The cushion pad according to claim 36, wherein the pad comprises an embossed spacer fabric material.

60. The cushion pad according to claim 36, wherein the pad comprises a sack filled with a flowable material.

61. The cushion pad according to claim 36, wherein the pad comprises at least one of moisturizers or healing agents, phase changing material, an antibacterial, an anti-wicking agent, or a vapor transfer material.

62. The cushion pad according to claim 36, wherein the pad is electrostatic.

63. The cushion pad according to claim 36, wherein the pad comprises a dual layer material.

64. The cushion pad according to claim 36, wherein the pad is thermoformed, molded, machined, stitched, die cut and/or compression cut.

65. A mask comprising:
a cushion and the cushion pad of claim 36,
wherein the cushion has a first portion to form a seal with the patient's skin, and a second portion to seal with the cushion pad in use.

66-83. (canceled)
